(19) United States
(12) Patent Application Publication
(10) Pub. No.: US 2019/0060325 A1
(43) Pub. Date: Feb. 28, 2019

(54) STABILIZED SOLID DOSAGE FORM

(71) Applicant: SHIONOGI & CO., LTD., Osaka-shi, Osaka (JP)

(72) Inventors: Go KIMURA, Amagasaki-shi, Hyogo (JP); Akira KAWAI, Amagasaki-shi, Hyogo (JP); Takeshi SAKAI, Osaka-shi, Osaka (JP)

(21) Appl. No.: 16/091,006

(22) PCT Filed: Apr. 7, 2017
(86) PCT No.: PCT/JP2017/014500

§ 371 Date: Oct. 3, 2018

(30) Foreign Application Priority Data
Apr. 8, 2016 (JP) 2016-078173

(51) Int. Cl. A61K 31/541; A61K 47/12; A61K 9/20

(52) U.S. Cl. CPC A61K 31/541; A61K 47/12; A61K 9/2054; A61K 9/2018; A61K 9/2095

(57) ABSTRACT

The present invention provides a solid dosage form suppressing the increase of by-products due to dimerization of a drug compound having an amino group and a cyano group. The solid dosage form of the invention is prepared by a process which comprises a process wherein a granulation liquid comprising a carboxylic acid ester is added to granulation powders comprising a compound having an amino group and a cyano group.

### FIELD

The present invention relates to a stabilized solid dosage form.

### BACKGROUND

A wide variety of additives has been investigated for stabilizing solid dosage forms. For example, it has been reported that the preservation stability was improved by addition of triethyl citrate (Patent literatures 3, 4 and 5). Also, there is a report that certain flavor was stabilized by addition of a Miglyol (Patent literature 6). Furthermore, it has been reported that addition of Polysorbate 80 (Tween 80) improved the elution of certain compound (Patent literature 7).

### CITATION LIST

### Patent Literature

### [PTL 1]

### WO 2009/151098

### [PTL 2]

### WO 2011/071057

### [PTL 3]

### JP-A-2013-14547

### [PTL 4]

### JP-A-2013-121951

### [PTL 5]

### JP-A-2015-180684

### [PTL 6]

### JP-A-2015-514713

### [PTL 7]

### JP-A-2015-510180

### SUMMARY

### Technical Problem

Certain drug compounds may involve deterioration of the drug purity because of dimerization of the compound in a pharmaceutical dosage form during process for the production and/or storage thereof.

The compound of formula (IV):

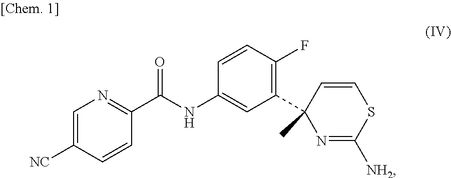

which may be hereinafter referred to as “compound (IV)”, has an effect on the prevention of beta-amyloid production, which is a main cause of Alzheimer's disease (AD), by inhibiting beta-secretase. Thus, it has been expected as a therapeutic agent for AD (Patent literatures 1 and 2). However, during the investigation for pharmaceutical dosage form of this compound, it has been found a problem that the drug purity is deteriorated with increasing of by-products due to dimerization of the compound.

The present invention provides an additive for suppressing the increase of by-products due to such dimerization of the compound (IV) in a solid dosage form and also provides a stable oral dosage form for this compound. Furthermore, the present invention provides an additive for suppressing the increase of by-products due to dimerization of a drug compound, which is not limited to the compound (IV), in a solid dosage form.

### Solution to Problem

The solid dosage form provided by the present invention includes:

[1] A solid dosage form containing a compound having an amino group and a cyano group, its pharmaceutically acceptable salt or a solvate thereof, and a carboxylic acid ester;
[2] The solid dosage form of [1] wherein the amino group is a reactive amino group and the cyano group is a reactive cyano group;
[3] The solid dosage form of [1] wherein the compound having an amino group and a cyano group has a group of formula (I):

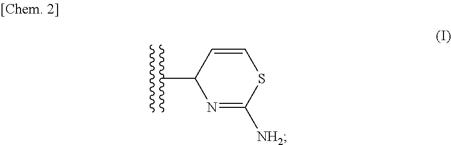

and
a group of formula (II):

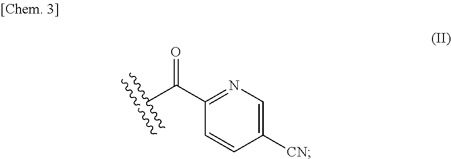

[4] The solid dosage form of [1] wherein the compound having an amino group and a cyano group is a compound of formula (III):

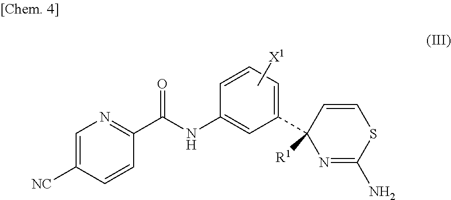

wherein
X¹ is halogen, and R¹ is an optionally substituted alkyl;
[5] The solid dosage form of [1] wherein the compound having an amino group and a cyano group is a compound of formula (IV):

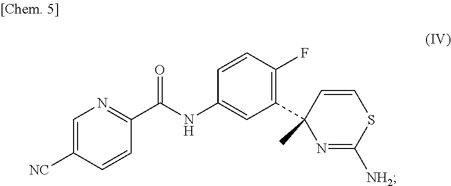

[6] The solid dosage form of any one of [1] to [5] wherein the carboxylic acid ester is one or more independently selected from the group consisting of polyvalent carboxylic acid esters, polyhydric alcohol esters and polyoxyethylene ethers of polyhydric alcohol ester;
[7] The solid dosage form of any one of [1] to [5] wherein the carboxylic acid ester is one or more independently selected from the group consisting of esters of citric acid, esters of glycerol, and polyoxyethylene ethers of sorbitan ester;
[8] The solid dosage form of any one of [1] to [5] wherein the carboxylic acid ester is one or more independently selected from the group consisting of lower alkyl esters of citric acid, medium-chain fatty acid esters of glycerol, short-chain fatty acid esters of glycerol, and polyoxyethylene ethers of sorbitan fatty acid ester;
[9] The solid dosage form of any one of [1] to [5] wherein the carboxylic acid ester is one or more independently selected from the group consisting of triethyl citrate, miglyol, polyoxyethylene sorbitan oleate and triacetin;
[10] The solid dosage form of any one of [1] to [5] wherein the carboxylic acid ester is triethyl citrate;
[11] The solid dosage form of any one of [1] to [10] wherein the dosage form contains one or more pharmaceutically acceptable excipient(s) selected from the group consisting of fillers, binders, disintegrants and lubricants;
[12] The solid dosage form of any one of [1] to [11] wherein the dosage form contains one or more selected from the group consisting of D-mannitol, lactose, low-substituted hydroxypropyl cellulose, croscarmellose sodium, hydroxypropyl cellulose and magnesium stearate;
[13] The solid dosage form of any one of [1] to [12] wherein the dosage form is substantially free of crystalline cellulose as excipient;
[14] The solid dosage form of any one of [1] to [13] wherein the dosage form is a tablet;
[15] A solid dosage form containing a compound of formula (IV):

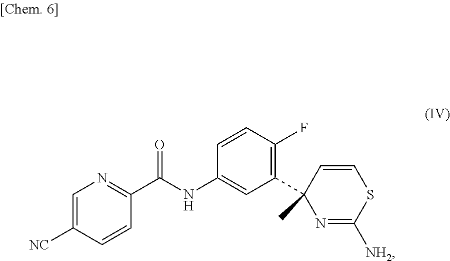

its pharmaceutically acceptable salt or a solvate thereof, and one or more pharmaceutically acceptable additive(s), which is substantially free of crystalline cellulose as excipient;
[16] A process for the production of the solid dosage form of any one of claims [1] to [14] which comprises a process wherein a granulation liquid comprising a carboxylic acid ester is added to granulation powders comprising a compound having an amino group and a cyano group or a pharmaceutically acceptable salt or a solvate thereof;
[17] A method of stabilizing a compound having an amino group and a cyano group, its pharmaceutically acceptable salt or a solvate thereof in a solid dosage form by adding a carboxylic acid ester;
[18] A method of preventing dimerization of a compound having an amino group and a cyano group, its pharmaceutically acceptable salt or a solvate thereof in a solid dosage form by adding a carboxylic acid ester;
[19] A solid dosage form containing a compound of formula (IV):

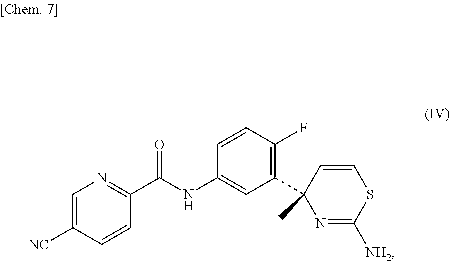

its pharmaceutically acceptable salt or a solvate thereof, and one or more related substance(s), wherein the relative amount of each individual related substance(s) is less than or equal to 0.25% of % peak area of HPLC;
[20] A solid dosage form containing a compound of formula (IV):

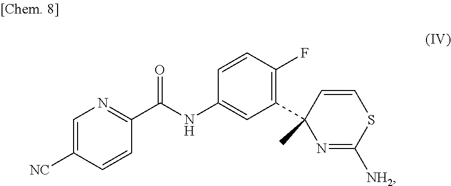

its pharmaceutically acceptable salt or a solvate thereof, and one or more related substance(s), wherein the total relative amount of individual related substance(s) is less than or equal to 0.65% of % peak area of HPLC.

The inventors prepared tablets of the compound (IV) in combination with various additives and investigated for suppression of by-products due to dimerization of the compound (IV). Surprisingly, the inventors have found that certain liquid additives have an effect on the suppression of such by-products.

Furthermore, the inventors have found a mechanism of suppression of such by-products due to dimerization based on the interaction between the liquid additives of the invention and the compound (IV). Accordingly, the effect of the invention is not limited to the compound (IV), and the liquid additive of the invention has an effect on suppression of by-products due to dimerization of other compounds having similar chemical structure.

### BRIEF DESCRIPTION OF DRAWINGS

FIG. 1 represents the infrared absorption spectrum of the mixture of compound (IV) and triethyl citrate.

FIG. 2 represents the results of storage stability test of the tablets of Example 1 containing triethyl citrate (“Containing TEC”) and Comparative Example 1 without triethyl citrate (“Without TEC”).

### DESCRIPTION OF EMBODIMENTS

The following is detailed description of the invention.

The drug compound of the invention is a compound that may dimerize, and thus, cause deterioration of the drug purity in the dosage form during a process for the production and/or storage thereof. Specifically, such drug compound may be a compound having an amino group and a cyano group in the molecule and may cause dimerization by the reaction of these groups. In one embodiment, the amino group is a reactive amino group. In one embodiment, the cyano group is a reactive cyano group.

Examples of such compound include a compound of formula (III):

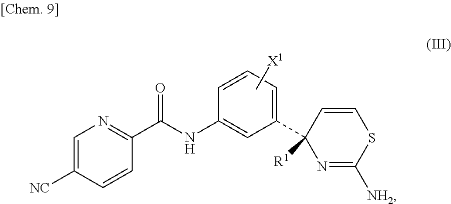

wherein X¹ is halogen, and R¹ is an optionally substituted alkyl. In this case, a compound of formula (III) has

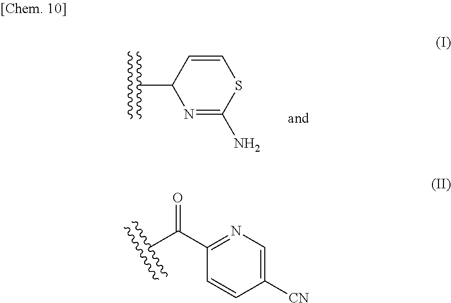

as a reactive amino group and a reactive cyano group, respectively, and it is predicted that the amino group of one compound is reacted with the cyano group of the other compound to form a dimer.

The preparation of Compound (IV) is shown in WO 2009/151098.

The respective terms used herein are as defined alone or in combination with other terms as follows.

The term “halogen” includes fluorine, chlorine, bromine and iodine. Preferably, “halogen” for X¹ is fluorine. Preferably, “halogen” for X² is bromine.

The term “alkyl” includes straight or branched alkyls of a carbon number of 1 to 8, preferably 1 to 6, and further preferably 1 to 3, and examples include methyl, ethyl, n-propyl, isopropyl, n-butyl, isobutyl, sec-butyl, tert-butyl, n-pentyl, isopentyl, neopentyl, hexyl, isohexyl, n-heptyl, isoheptyl, and n-octyl.

Examples of the substituent of “optionally substituted alkyl” include same or different one or more group(s), preferably 1 to 3 group(s) selected from halogen such as fluorine. Examples of “optionally substituted alkyl” are methyl, fluoromethyl, difluoromethyl and trifluoromethyl.

The salts of the compounds according to the present invention include, for example, salts with alkaline metal (e.g., lithium, sodium, potassium or the like), alkaline earth metal (e.g., calcium, barium or the like), magnesium, transition metal (e.g., zinc, iron or the like), ammonia, organic bases (e.g., trimethylamine, triethylamine, dicyclohexylamine, ethanolamine, diethanolamine, triethanolamine, meglumine, ethylenediamine, pyridine, picoline, quinoline or the like) or amino acids, or salts with inorganic acids (e.g., hydrochloric acid, sulfuric acid, nitric acid, carbonic acid, hydrobromic acid, phosphoric acid, hydroiodic acid or the like) or organic acids (e.g., formic acid, acetic acid, propionic acid, trifluoroacetic acid, citric acid, lactic acid, tartaric acid, oxalic acid, maleic acid, fumaric acid, mandelic acid, glutaric acid, malic acid, benzoic acid, phthalic acid, ascorbic acid, benzenesulfonic acid, p-toluenesulfonic acid, methanesulfonic acid, ethanesulfonic acid or the like). Especially, salts with hydrochloric acid, sulfuric acid, phosphoric acid, tartaric acid, methanesulfonic acid and the like are included. These salts can be formed by the usual methods. The salts of the compound (IV) include salts with the above inorganic acids or the above organic acids.

The compounds according to the present invention or salts thereof may form solvates, such as hydrates or the like, cocrystal and/or crystal polymorphs. The compounds according to the present invention encompass those various solvates, cocrystal and crystal polymorphs. “Solvates” may be those wherein any numbers of solvent molecules, such as water molecules or the like, are coordinated with the compounds. When the compounds or salts thereof are allowed to stand in the atmosphere, the compounds may absorb water, resulting in attachment of adsorbed water or formation of hydrates. Recrystallization of the compounds or salts thereof may produce crystal polymorphs. The term “cocrystal” means that a compound or salt thereof and a counter-molecule exist in the same crystal lattice, and it can be formed with any number of counter-molecules.

The following Scheme 1 describes an exemplary process of the invention to prepare Compound (III).

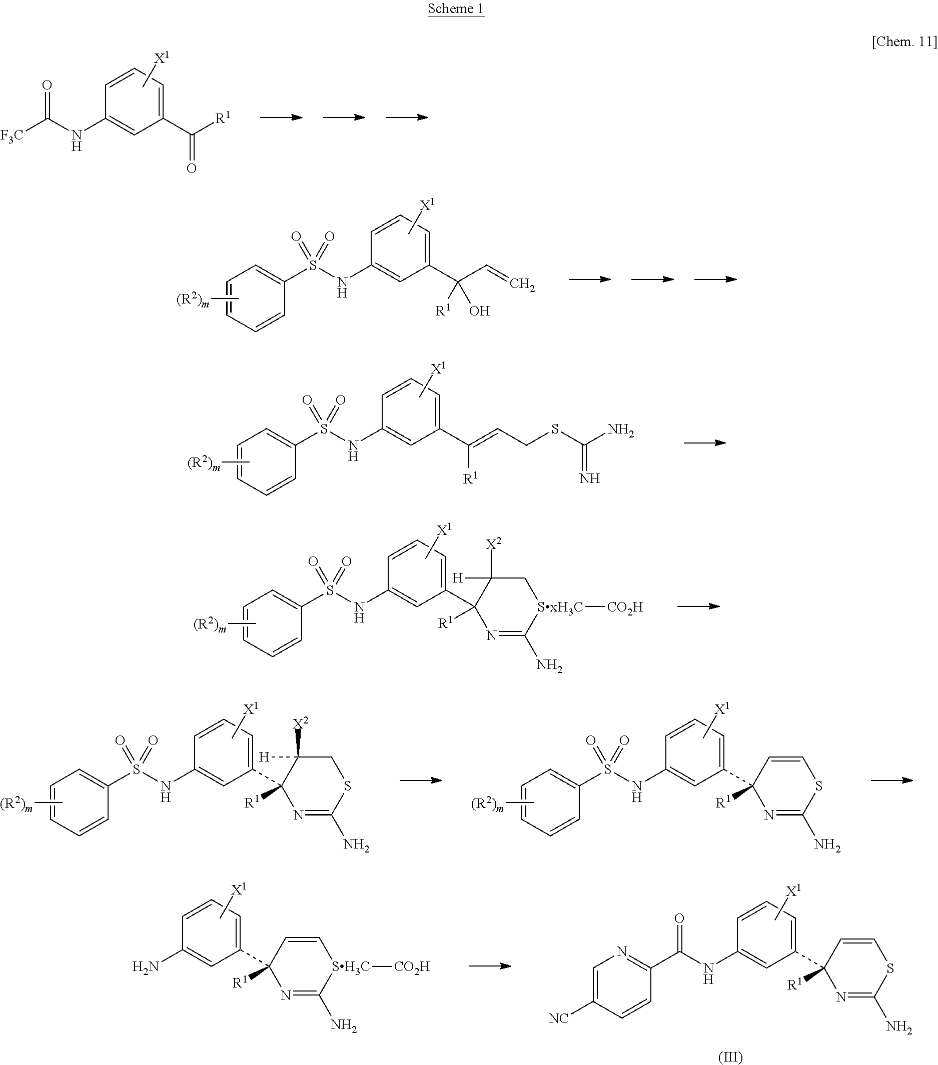

wherein X¹ is halogen, X² halogen, R¹ is an optionally substituted alkyl, R² is nitro, trifluoromethyl, methoxy, or bromo and m is an integer of 0 to 2.

During storage of the solid dosage form of the compound (IV), related substances derived from the compound (IV) are formed. Such related substances as typically found include five substances, which are herein referred to as the related substances A, B, C, D and E. The related substance C is a dimer of the compound (IV). The respective masses (m/z) thereof are 769.1751 (A and B), 735.1874 (C), 735.1871 (D) and 498.1 (E). The mass (m/z) of the compound (IV) is 368.1.

The solid dosage form of the invention is stable during storage thereof. For example, after the storage at 60° C. for two months, each relative amount of said related substances A to E is less than or equal to 0.25% of % peak area, and the total relative amount of these related substances is less than or equal to 0.65% of % peak area, when determined by HPLC method under the condition described below in the working example.

Even for the related substance C, which is a dimer of the compound (IV) and particularly tends to increase during the storage, the relative amount thereof is not greater than 0.25% of peak area of HPLC.

The inventors have found that addition of triethyl citrate suppressed the increase of related substance C in a solid dosage form of the compound (IV). Also, the interaction between the ester functional group in triethyl citrate and the amino group in the compound (IV) was found (see, [0055]). This indicates that the liquid additive of the invention interacts with the amino group of the compound (IV), thereby the side-reaction between the amino group and the cyano group of the compound to form related substance C is inhibited.

The liquid additive of the invention that suppresses the increase of by-products due to dimerization has an ester functional group. Examples of such liquid additive include carboxylic acid esters. Examples of such carboxylic acid esters include polyvalent carboxylic acid esters, polyhydric alcohol esters and polyoxyethylene ethers of polyhydric alcohol ester.

Examples of the polyvalent carboxylic acid ester include esters of citric acid. Examples of the polyhydric alcohol ester include esters of glycerol. Examples of the polyoxyethylene ether of polyhydric alcohol ester include polyoxyethylene ethers of sorbitan ester.

Examples of the ester of citric acid include lower alkyl esters of citric acid. Examples of the ester of glycerol include medium-chain fatty acid esters of glycerol and short-chain fatty acid esters of glycerol. Examples of the polyoxyethylene ether of sorbitan ester include polyoxyethylene ethers of sorbitan fatty acid ester.

Examples of the lower alkyl esters of citric acid include triethyl citrate (TEC). Examples of the medium-chain fatty acid esters of glycerol include miglyol, such as Miglyol 812. Examples of the short-chain fatty acid esters of glycerol include triacetin. Examples of the polyoxyethylene ethers of sorbitan fatty acid ester include polyoxyethylene sorbitan oleate, such as Polysorbate 80 (Tween80).

The technique according to the present invention using a liquid additive of the invention for suppressing the increase of the related substance can be applied generally to any solid dosage form.

Examples of the dosage form of the solid dosage form of the invention include oral dosage forms, such as tablets including sublingual and orally-disintegrating tablets, capsules including soft capsules and microcapsules, powders, granules, pellets and troches; parenteral dosage forms, such as topical dosage forms including transdermal preparations and ointments, suppositories including rectal and vaginal suppositories. These dosage forms may be rapid-release dosage forms or controlled-release dosage forms such as sustained-release dosage forms.

The technique according to the invention for suppressing the increase of related substances can be applied preferably to oral dosage forms, especially tablets.

Furthermore, the technique for suppressing the increase of related substances according to the present invention can be applied to a process for the production of a solid dosage form comprising a drug compound having an amino group and a cyano group as an active ingredient. For example, in case of a tablet, pharmaceutically acceptable additives conventionally used for tableting include excipients such as fillers, binders, disintegrants and lubricants are added to the active ingredient and mixed them to obtain a granulation powder, followed by addition of a granulation liquid comprising a liquid additive of the invention and subjecting the mixture to a granulation process according to conventional methods. The granules thus obtained in the granulation process may be conventional methods to obtain a tablet.

In additional embodiment, the present invention provides a method for stabilizing a compound having an amino group and a cyano group or a pharmaceutically acceptable salt or a solvate thereof in a solid dosage form. The method comprises steps wherein pharmaceutically acceptable additives conventionally used for tableting such as excipients of fillers, binders, disintegrants and lubricants are added to the active ingredient and mixed them to obtain a granulation powder, followed by addition of a granulation liquid comprising a liquid additive of the invention and subjecting the mixture to a granulation process according to conventional methods.

In additional embodiment, the present invention provides a method for suppressing dimerization of a compound having an amino group and a cyano group or a pharmaceutically acceptable salt or a solvate thereof in a solid dosage form. The method comprises process wherein pharmaceutically acceptable additives conventionally used for tableting such as excipients of fillers, binders, disintegrants and lubricants are added to the active ingredient and mixed them to obtain a granulation powder, followed by addition of a granulation liquid comprising a liquid additive of the invention and subjecting the mixture to a granulation process according to conventional methods.

As described, the additive of the present invention is added as a liquid to a granulation powder of a drug compound. This makes the additive of the invention be in contact homogeneously with the drug compound, and therefore, is effective in suppressing the dimerization of the drug compound.

The amount of the compound (IV) is usually from 0.1 to 50% by weight, preferably from 0.5 to 25% by weight and more preferably from 2.5 to 20% by weight of the dosage form.

The amount of the liquid additive of the present invention is usually from 0.05 to 6% by weight, and preferably from 0.075 to 2.5% by weight and more preferably from 0.1 to 1.75% by weight of the dosage form.

Examples of the filler include D-mannitol, corn starch, lactose, purified sucrose, maltitol and anhydrous sodium hydrogen phosphate. Among others, D-mannitol is preferable. The amount of the excipients is from 10 to 99.9% by weight, preferably from 35 to 95% by weight, and more preferably from 50 to 90% by weight of the dosage form. The amount of the excipients is from 0.01 to 0.2 moles, preferably from 0.02 to 0.175 moles, and more preferably from 0.03 to 0.15 moles, per 1 mol of the compound (IV).

Examples of the binder include hydroxypropyl cellulose and partly pregelatinized starch. Among others, hydroxypropyl cellulose is preferable. The amount of the binder is from 1 to 30% by weight, preferably from 1.5 to 20% by weight, and more preferably from 2 to 10% by weight of the dosage form.

Examples of the disintegrant include low-substituted hydroxypropyl cellulose, carmellose calcium, croscarmellose sodium and sodium carboxymethyl starch. Among others, low-substituted hydroxypropyl cellulose and sodium carboxymethyl starch are preferable. The amount of the disintegrant is from 1 to 30% by weight, more preferably from 2 to 25% by weight, and more preferably from 3 to 20% by weight of the dosage form.

Examples of the lubricant include sodium stearyl fumarate and magnesium stearate. Among others, magnesium stearate is preferable. The amount of the lubricant is from 0.5 to 2% by weight, preferably from 0.5 to 1.75% by weight, and more preferably from 0.5 to 1.5% by weight of the dosage form.

In one embodiment of the invention, the solid dosage form of the invention is substantially free of crystalline cellulose as excipient. As used herein, the term “substantially free of crystalline cellulose as excipient” means that the solid dosage form of the invention may not contain crystalline cellulose in an amount that serves as an excipient, such as less than 1% by weight, preferably less than 0.5% by weight, and more preferably less than 0.25% by weight.

The uncoated tablets obtained as described above may be film-coated as necessary. Examples of the coating base used for such film-coating include sugar coating bases, water-soluble film coating bases, enteric film coating bases and sustained-release film coating bases.

Examples of the sugar coating base include sucrose, which may be used in combination with one or two materials selected from talc, precipitated calcium carbonate, gelatin, gum arabic, pullulan and carnauba wax.

Examples of the water-soluble film coating base include cellulosic polymers such as hydroxypropyl celluloses [e.g., Grade: L, SL, SL-T, SSL (trade name); NIPPON SODA CO., LTD.], hydroxypropyl methylcelluloses [e.g., hypromellose 2910, TC-5 (Grade: MW, E, EW, R and RW) (trade name); Shin-Etsu Chemical Co., Ltd.], hydroxyethyl celluloses and methyl hydroxyethyl celluloses; synthetic polymers such as polyvinyl acetal diethyl amino acetate, aminoalkylmethacrylate copolymer E [EUDRAGIT E (trade name)] and polyvinylpyrrolidone; and polysaccharides such as pullulan.

Examples of the enteric film coating base include cellulosic polymers such as hydroxypropyl methylcellulose phthalate, hydroxypropyl methylcellulose acetate succinate, carboxy methyl ethyl cellulose and cellulose acetate phthalate; acrylic polymers such as methacrylic acid copolymer L [EUDRAGIT L (trade name)], methacrylic acid copolymer LD [EUDRAGIT L-30D55 (trade name)] and methacrylic acid copolymer S [EUDRAGIT S (trade name)]; and natural products such as shellac.

Examples of the sustained-release film coating base include cellulosic polymers such as ethyl cellulose; and acrylic polymers such as aminoalkylmethacrylate copolymer RS [EUDRAGIT RS (trade name)], and ethyl acrylate and methyl methacrylate copolymer dispersion [EUDRAGIT NE (trade name)].

In case where the uncoated tablets are film coated, the film coating layer is usually from 1 to 10 parts by weight, preferably from 2 to 6 parts by weight, per 100 parts by weight of the uncoated tablets.

The present invention shall be explained in more detail in the following examples with reference to the figures, but without being limited thereto.

### EXAMPLES

### Effect of Excipients on Suppression of Related Substance Formation

Effect of excipients on suppression of related substance formation was examined. The excipients were physically (dry) mixed or were wet-kneaded using purified water with the compound (IV) in designated ratios in Table 1. The wet-kneaded materials were dried at 60° C. with ventilation. The dried materials were subdivided in glass bottles and the bottles were capped. The capped samples were stored at 60° C. for two weeks and the effect on suppression of related substance formation was estimated by the increased amount of related substances.

### Preparation of Related Substances

10 g of the compound (IV) was stored at 60° C. for three weeks and purified sequentially by fractional recrystallization, and normal and reverse phase chromatography to isolate the related substance A (33.83 mg), B (9.36 mg) and D (4.26 mg). Separately, 2.5 g of spray-dried compound (IV) was stored at 60° C. for two weeks and purified by reverse phase chromatography to isolate the related substance C (26.42 mg). The related substance E was isolated during the process for the preparation of compound (IV).

### Measurement of Related Substances

The relative amounts of the related substances in a 25 mg tablet obtained in the following Examples were determined by HPLC under the following conditions.

Detector: Ultraviolet spectrophotometer (wavelength: 245 nm)

### Column: Unison UK-C18, 3 μm, 4.6×100 mm (Imtakt)

Column temperature: 40° C.
Solution A: 0.1% trifluoroacetic acid solution

### Solution B: Acetonitrile

### Gradient Program:

Table 1 shows the time table on the flow of solution A and solution B

TABLE 1

| Time (min) | Solution A (%) | Solution B (%) |
|---|---|---|
| 0-12 | 78 | 22 |
| 1-35 | 78 → 40 | 22 → 60 |
| 35-45 | 40 | 60 |
| 45-45.01 | 40 → 78 | 60 → 22 |
| 45.01-60 | 78 | 22 |

Flow rate: About 1.0 mL/min

### Retention Time:

Compound (IV): 9 to 10 min;
Related substance A: 20 to 21 min;
Related substance B: 21 to 22 min;
Related substance C: 22 to 23 min;
Related substance D: 28 to 29 min;
Related substance E: 36 to 37 min.
   Injection volume: 10 μL
   Analytical time: 45 min

Related substances of compound (IV) were calculated by the following equation.

$$\begin{matrix}Relative amount of compound ( IV ) individual related substance = \frac{Ai}{Σ Ai + As} \times 100 & [ Math . 1 ] \\ Total relative amount of compound ( IV ) related substance = \frac{Σ Ai}{Σ Ai + As} \times 100 & [ Math . 2 ]\end{matrix}$$

As: Peak area of compound (IV) in a standard solution
Ai: Peak area of individual related substance
ΣAi: Sum of peak area of individual related substance

Table 2 shows the effect of excipients on suppression of related substance C formation.

TABLE 2

| | Ratio | Relative amount (%) of |
|---|---|---|
| | (compound | related substance C after |
| Excipients | (IV):Excipients) | storage of 60° C./2 W |
| N.A. | compound (IV) only | 0.19 |
| | milled | (Initial: 0.05%) |
| N.A. | compound (IV) only | 0.16 |
| | (Wet-kneaded) | |
| Filler | | |
| D-Mannitol | 1:10 | 0.15 |
| Corn starch | 1:10 | 0.23 |
| Microcrystalline | 1:10 | 0.48 |
| cellulose | | |
| Lactose | 1:10 | 0.20 |
| Maltitol | 1:10 | 0.22 |
| Anhydrous dibasic | 1:10 | 0.24 |
| calcium phosphate | | |
| Binder | | |
| Hydroxypropyl | 3:1 | 0.23 |
| cellulose | | |
| Pregelatinized starch | 3:1 | 0.24 |
| Low-substituted | 3:1 | 0.28 |
| hydroxypropyl | | |
| cellulose | | |
| Disintegrant | | |
| Carmellose calcium | 2:1 | 0.37 |
| Croscarmellose | 2:1 | 0.34 |
| sodium | | |
| Sodium starch | 2:1 | 0.27 |
| glycolate | | |
| Lubricant | | |
| Sodium stearyl | 10:1 | 0.16 |
| fumarate | (Dry-mixed) | |
| Magnesium stearate | 10:1 | 0.16 |
| | (Dry-mixed) | |
| Gradient | | |
| Talc | 10:1 | 0.17 |
| | (Dry-mixed) | |
| bFilm coating agent | | |
| Hypromellose | 5:1 | 0.22 |
| Titanium dioxide | 10:1 | 0.14 |
| Triethyl citrate | 10:1 | 0.10 |

During the test, the excipients listed in Table 2 did not cause significant increase of the amount of the related substances A, B, D and E, and the relative amounts of these related substances were less than or equal to 0.25% of % peak area of HPLC. On the other hand, as shown in Table 2, some excipients provided increased amounts greater than 0.25% of % peak area of HPLC of the related substance C. The relative amount of the related substance C in the mixture containing crystalline cellulose and compound (IV) was increased to 0.48% of % peak area of HPLC after storage at 60° C. for two weeks. On the other hand, the relative amount of the related substance C in the mixture containing D-Mannitol as a filler, sodium starch glycolate and low-substitute hydroxypropyl cellulose as a disintegrant, hydroxypropyl cellulose as a binder, magnesium stearate as lubricant for the core tablet, and hypromellose, talc, titanium dioxide and triethyl citrate as film coating agents, and compound (IV) was lower. Especially, the relative amount of related substance C in the mixture containing triethyl citrate was 0.10% of % peak area after storage at 60° C. for two weeks.

### Effect of Liquid Additives on Suppression of Related Substance Formation

Effect of liquid additives on suppression of related substance formation was examined. The liquid additives were wet-kneaded using purified water with the drug substance in designated ratios in the Table 3. The amount of TEC was shown at the mole ratios to compound (IV) in this Table 3. The wet-kneaded materials were dried at 60° C. with ventilation. The dried materials were subdivided in glass bottles and the bottles were capped. The capped samples were stored at 60° C. for two weeks.

TABLE 3

| | Mole ratio to |
|---|---|
| Liquid additives | compound (IV) |
| Triethyl citrate | 0.05 |
| Triethyl citrate | 0.13 |
| Tri (capryl•capric acid) glyceryl (Mygriol 812) | 0.1 |
| Polyoxyethylene Sorbitan Monooleate (Tween 80) | 0.1 |
| Polyoxyethylene (20) polyoxypropiren (20) glycol | 0.1 |
| (Lutrol L-44) | |

Effect of liquid additives on suppression of related substance C is shown in Table 4. The relative amount of related substance C in the mixture containing polyoxyethylene (20) polyoxypropiren (20) glycol and compound (IV) was increased up to 0.2% of % peak area of HPLC after storage at 60° C. for two weeks, while the relative amount of related substance was less than 0.19% of % peak area of HPLC in the mixture containing triethyl citrate, tri(capryl.capric acid)glyceryl, polyoxyethylene sorbitan monooleate and compound (IV). Triethyl citrate, tri(capryl.capric acid)glyceryl and polyoxyethylene sorbitan monooleate are carboxylic acid ester. The relative amounts of the related substances A, B, D and E were less than or equal to 0.25% of % peak area of HPLC and not increased significantly during the test.

TABLE 4

| | | Relative amount (%) of |
|---|---|---|
| | Mole ratio to | related substance C after |
| Liquid additives | compound (IV) | storage of 60° C./2 W* |
| Triethyl citrate | 0.05 | 0.10 |
| Triethyl citrate | 0.13 | 0.10 |
| Tri (capryl•capric acid) | 0.1 | 0.09 |
| glyceryl (Mygriol 812) | | |
| Polyoxyethylene Sorbitan | 0.1 | 0.15 |
| Monooleate (Tween 80) | | |
| Polyoxyethylene (20) | 0.1 | 0.20 |
| polyoxypropiren (20) | | |
| glycol (Lutrol L-44) | | |

*0.05% at the start of the test

### Interaction of Triethyl Citrate

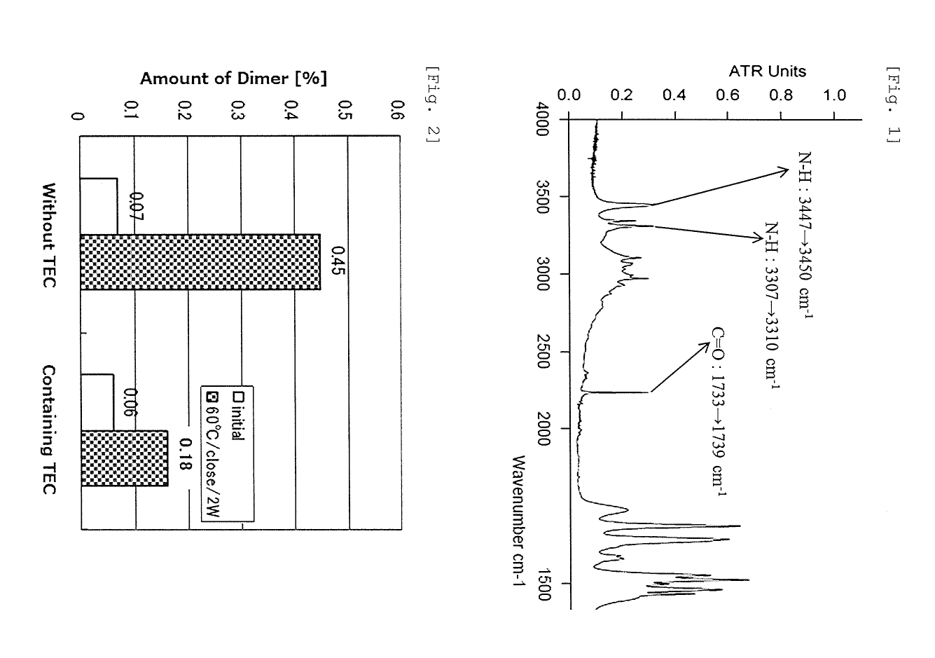

A mixture of compound (IV) and triethyl citrate was analyzed by infrared absorption spectroscopy. The infrared absorption spectrum is shown in FIG. 1. Comparing with the infrared absorption spectrum for each of compound (IV) and triethyl citrate (not shown), significantly shifts for the absorption of N—H of the compound (IV) and the absorption of C═O of triethyl citrate (3 cm⁻¹ and 6 cm⁻¹, respectively) were observed, indicating the interaction between the amino group of compound (IV) and the ester functional group of triethyl citrate. Thus, it is concluded that the amino group of compound (IV) and the ester functional group of triethyl citrate are interacted.

### Example 1

According to the regimen of Table 5, the compound (IV), D-mannitol and Low-substituted hydroxypropyl cellulose were mixed homogeneously to obtain a granulation powder. The granulation powder was granulated according to conventional method, by adding 1.9 mg or 9.4 mg of triethyl citrate in hydroxypropyl cellulose solution as a granulation liquid. The granules thus obtained were compressed to according to conventional method after mixing with sodium carboxymethyl starch and magnesium stearate to give 60 tablets containing 25 mg of the compound (IV) per tablet. The core tablets were compressed at 5 kN.

For Comparative Example 1, all the components except triethyl citrate were combined and tableted in the same manner to obtain a tablet.

TABLE 5

mg/tablet

| | Comparative | | Comparative |
|---|---|---|---|
| Components | Example 1 | Example 1 | Example 2 |
| Compound (IV) | 25 | 25 | 25 |
| Triethyl citrate | — | 1.9 | 9.4 |
| D-mannitol | 99.5 | 97.6 | 90.1 |
| Low-substituted | 15 | 15 | 15 |
| hydroxypropyl | | | |
| cellulose | | | |
| Hydroxypropyl | 4.5 | 4.5 | 4.5 |
| cellulose | | | |
| Sodium | 4.5 | 4.5 | 4.5 |
| carboxymethyl | | | |
| starch | | | |
| Magnesium stearate | 1.5 | 1.5 | 1.5 |
| Total | 150 | 150 | 150 |

### Effect on Hardness of Tablet

The average hardness of Example 1 (1.9 mg/tablet) and Comparative Example 2 (9.4 mg/tablet) was measured by hardness meter, and their hardness were 84 N (n=2) and 12 N (n=2), respectively. Hardness of 12 N was not acceptable and then it is concluded that mole ratio of triethyl citrate as stabilizer to compound (IV) should be 1.9 mg/tablet from the view of hardness.

### Effect on Suppression of Related Substance Formation

The tablet of Example 1 was compared with the tablet of Comparative Example 1 after storage at 60° C. for two weeks. The result is shown in FIG. 2. The relative amount of related substance C (“Dimer”) in the tablet of Comparative Example 1 containing no triethyl citrate was increased to 0.45% of % peak area of HPLC (Initial: 0.07%) after storage at 60° C. for two weeks, while the relative amount was 0.18% of % peak area of HPLC (Initial: 0.06%) in the tablet of Example 1. The relative amounts of the related substances A, B, D and E were less than or equal to 0.25% of % peak area of HPLC and not increased significantly during the test.

### Examples 2 and 3

According to the regimen of Table 6, the compound (IV), D-mannitol and Low-substituted hydroxypropyl cellulose were mixed homogeneously to obtain a granulation powder. The granulation powder was granulated according to conventional method, by adding 0.4 mg or 1.9 mg of triethyl citrate in hydroxypropyl cellulose solution as a granulation liquid. The granules thus obtained were compressed to according to conventional method after mixing with sodium carboxymethyl starch and magnesium stearate to give 60 tablets containing 5 mg of the compound (IV) per tablet. The core tablets were compressed at 5 kN. For Comparative Example 3, all the components except triethyl citrate were combined and tableted in the same manner to obtain a tablet.

TABLE 6

| | mg/tablet | | |
|---|---|---|---|
| | Comparative | | |
| Components | Example 3 | Example 2 | Example 3 |
| Compound (IV) | 5 | 5 | 5 |
| Triethyl citrate | — | 0.4 | 1.9 |
| D-mannitol | 119.5 | 119.1 | 117.6 |
| Low-substituted | 15 | 15 | 15 |
| hydroxypropyl | | | |
| cellulose | | | |
| Hydroxypropyl | 4.5 | 4.5 | 4.5 |
| cellulose | | | |
| Sodium | 4.5 | 4.5 | 4.5 |
| carboxymethyl | | | |
| starch | | | |
| Magnesium stearate | 1.5 | 1.5 | 1.5 |
| Total | 150 | 150 | 150 |

### Effect on Suppression of Related Substance Formation

The tablets of Examples 2 and 3 were compared with the tablet of Comparative Example 3 after storage at 60° C. for two weeks. The result is shown in Table 7. The relative amount of the related substance C in the tablet of Comparative Example 3 containing no triethyl citrate was increased to 0.28% of % peak area of HPLC after storage at 60° C. for two weeks, while the relative amount was 0.15% and 0.12% of % peak area of HPLC in the tablets of Examples 2 and 3, respectively. The relative amounts of related substances A, B, D and E were less than or equal to 0.25% of % peak area of HPLC and not increased significantly during the test.

TABLE 7

| | Relative amount (%) of related substance C | | |
|---|---|---|---|
| | Comparative | | |
| Storage condition | Example 3 | Example 2 | Example 3 |
| Initial | 0.03 | 0.03 | 0.03 |
| 60° C. 2 weeks | 0.28 | 0.15 | 0.12 |

### INDUSTRIAL APPLICABILITY

The solid dosage form of the present invention is useful for reducing the amount of related substance, especially related substance C, which is a dimer of formula (IV).

1. A solid dosage form containing a compound having an amino group and a cyano group, its pharmaceutically acceptable salt or a solvate thereof, and a carboxylic acid ester.

2. The solid dosage form of claim 1 wherein the amino group is a reactive amino group and the cyano group is a reactive cyano group.

3. The solid dosage form of claim 1 wherein the compound having an amino group and a cyano group has a group of formula (I): 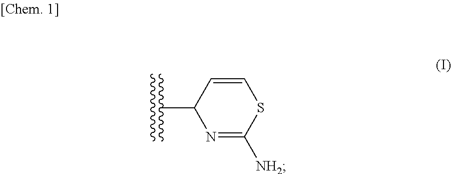
and
a group of formula (II):

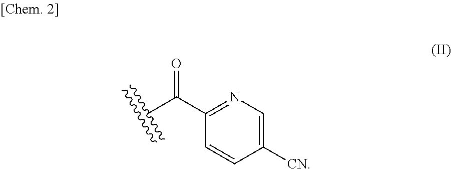

4. The solid dosage form of claim 1 wherein the compound having an amino group and a cyano group is a compound of formula (III): 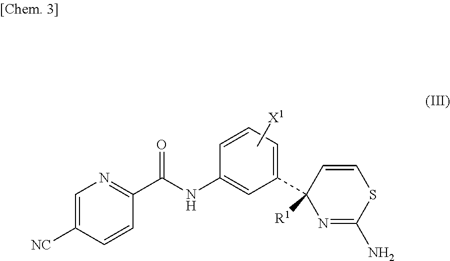
wherein
X¹ is halogen, and R¹ is an optionally substituted alkyl.

5. The solid dosage form of claim 1 wherein the compound having an amino group and a cyano group is a compound of formula (IV):

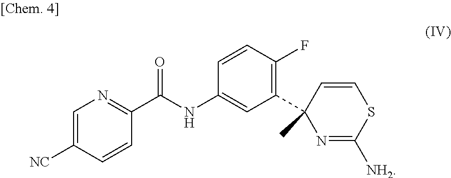

6. The solid dosage form of claim 1 wherein the carboxylic acid ester is one or more independently selected from the group consisting of polyvalent carboxylic acid esters, polyhydric alcohol esters and polyoxyethylene ethers of polyhydric alcohol ester.

7. The solid dosage form of claim 1 wherein the carboxylic acid ester is one or more independently selected from the group consisting of esters of citric acid, esters of glycerol, and polyoxyethylene ethers of sorbitan ester.

8. The solid dosage form of claim 1 wherein the carboxylic acid ester is one or more independently selected from the group consisting of lower alkyl esters of citric acid, medium-chain fatty acid esters of glycerol, short-chain fatty acid esters of glycerol, and polyoxyethylene ethers of sorbitan fatty acid ester.

9. The solid dosage form of claim 1 wherein the carboxylic acid ester is one or more independently selected from the group consisting of triethyl citrate, miglyol, polyoxyethylene sorbitan oleate and triacetin.

10. The solid dosage form of claim 1 wherein the carboxylic acid ester is triethyl citrate.

11. The solid dosage form of claim 1 wherein the dosage form contains one or more pharmaceutically acceptable excipient(s) selected from the group consisting of fillers, binders, disintegrants and lubricants.

12. The solid dosage form of claim 1 wherein the dosage form contains one or more selected from the group consisting of D-mannitol, lactose, low-substituted hydroxypropyl cellulose, croscarmellose sodium, hydroxypropyl cellulose and magnesium stearate.

13. The solid dosage form of claim 1 wherein the dosage form is substantially free of crystalline cellulose as excipient.

14. The solid dosage form of claim 1 wherein the dosage form is a tablet.

15. A solid dosage form containing a compound of formula (IV): 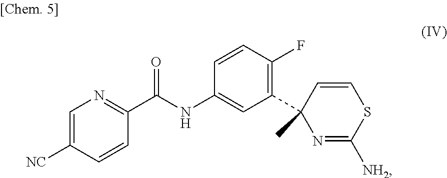
its pharmaceutically acceptable salt or a solvate thereof, and one or more pharmaceutically acceptable additive(s), which is substantially free of crystalline cellulose as excipient.

16. A process for the production of the solid dosage form of claim 1 which comprises a process wherein a granulation liquid comprising a carboxylic acid ester is added to granulation powders comprising a compound having an amino group and a cyano group or a pharmaceutically acceptable salt or a solvate thereof.

17. A method of stabilizing a compound having an amino group and a cyano group, its pharmaceutically acceptable salt or a solvate thereof in a solid dosage form by adding a carboxylic acid ester.

18. A method of preventing dimerization of a compound having an amino group and a cyano group, its pharmaceutically acceptable salt or a solvate thereof in a solid dosage form by adding a carboxylic acid ester.

19. A solid dosage form containing a compound of formula (IV): 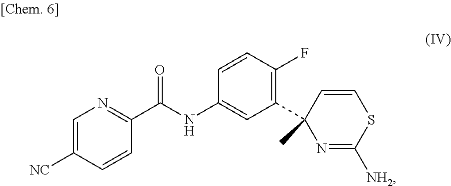
its pharmaceutically acceptable salt or a solvate thereof, and one or more related substance(s), wherein the relative amount of each individual related substance(s) is less than or equal to 0.25% of % peak area of HPLC.

20. A solid dosage form containing a compound of formula (IV): 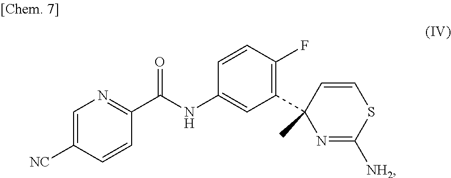
its pharmaceutically acceptable salt or a solvate thereof, and one or more related substance(s), wherein the total relative amount of individual related substance(s) is less than or equal to 0.65% of % peak area of HPLC.
